(19) United States
(12) Patent Application Publication
(10) Pub. No.: US 2025/0340468 A1
(43) Pub. Date: Nov. 6, 2025

(54) ACINETOBACTER PITTII STRAIN AND APPLICATION THEREOF

(71) Applicant: Yunnan Agricultural University, Kunming (CN)

(72) Inventors: Yishu DENG, Kunming (CN); Youbo SU, Kunming (CN); Jilai ZHANG, Kunming (CN); Yuming YIN, Kunming (CN); Ming JIANG, Kunming (CN); Jing WANG, Kunming (CN); Genhua YANG, Kunming (CN)

(21) Appl. No.: 19/267,575

(22) Filed: Jul. 13, 2025

(30) Foreign Application Priority Data
Feb. 12, 2025 (CN) 202510152295.2

(51) Int. Cl. C02F 3/34; C02F 101/10; C12N 1/20; C12R 1/01

(52) U.S. Cl. CPC C02F 3/34; C12N 1/205; C02F 2101/105; C12R 2001/01

(57) ABSTRACT

An Acinetobacter pittii strain is provided, which is Acinetobacter pittii FL3-2 with a deposit number of CCTCC NO: M20232618. A bacterial composition is provided, which includes the Acinetobacter pittii FL3-2 as an active component. A method for phosphorus removal from a sewage sample is also provided, in which the Acinetobacter pittii FL3-2 is inoculated into the to-be-treated sewage sample.

### REFERENCE TO AN ELECTRONIC SEQUENCE LISTING

The contents of the electronic sequence listing (Name: SequenceListing.xml; Size: 5,017 bytes; and Date of Creation: Jun. 25, 2025) is herein incorporated by reference in its entirety.

### CROSS-REFERENCE TO RELATED APPLICATIONS

This application claims the benefit of priority from Chinese Patent Application No. 202510152295.2, filed on Feb. 12, 2025. The content of the aforementioned application, including any intervening amendments made thereto, is incorporated herein by reference in its entirety.

### TECHNICAL FIELD

This application relates to biotechnology, and more particularly to an Acinetobacter pittii strain and an application thereof.

### BACKGROUND

Domestic sewage contains various harmful substances such as organic matter, inorganic matter, microorganisms and heavy metals. The direct discharge of the untreated sewage will severely threaten the ecological environment and human health. Particularly, excessive phosphorus level in the domestic sewage may cause eutrophication and thus endanger aquatic ecosystems and human health.

Phosphorus removal methods for domestic sewage mainly include physical treatment method, chemical treatment method and biological treatment method. The biological treatment method is mainly dependent on polyphosphate-accumulating organisms. The polyphosphate-accumulating organisms are a class of heterotrophic bacteria that can achieve anaerobic phosphorus release and aerobic excess phosphorus uptake through alternating between anaerobic and aerobic conditions. The metabolic mechanism involves utilizing stored carbon sources under the anaerobic condition and achieving excessive phosphorus uptake under the aerobic condition. The phosphorus removal efficiency is jointly determined by the proportion of the polyphosphate-accumulating organisms in the mixed microbial community and the excess phosphorus uptake capability of the polyphosphate-accumulating organisms. At present, the phosphorus removal ability of polyphosphate-accumulating organisms is susceptible to environmental factors such as pH, temperature and dissolved oxygen. In particular, the polyphosphate-accumulating organisms used for rural domestic sewage treatment in rural high-altitude mountainous areas are more sensitive to the environmental factors.

### SUMMARY

An object of the disclosure is to provide an Acinetobacter pittii strain and an application thereof, so as to solve the problems in the prior art. The Acinetobacter pittii strain provided herein is Acinetobacter pittii FL3-2, which has the phosphorus accumulation and removal capability.

In order to achieve the above object, the following technical solutions are adopted.

In a first aspect, this application provides an Acinetobacter pittii strain, wherein the Acinetobacter pittii strain is Acinetobacter pittii FL3-2 with a deposit number of CCTCC NO: M20232618.

In a second aspect, this application provides a bacterial composition, comprising:
the above Acinetobacter pittii strain as active ingredient.

In a third aspect, this application provides a method for phosphorus removal of a sewage sample, comprising:
inoculating the above Acinetobacter pittii strain or the above bacterial composition into the sewage sample.

Compared to the prior art, the present disclosure has the following beneficial effects.

In the present disclosure, a polyphosphate-accumulating strain, Acinetobacter pittii strain FL3-2, is isolated from the soil at Baima Snow Mountain (elevation of 4,000 m) in Deqin County, Diqing Prefecture, Yunnan Province. The Acinetobacter pittii strain FL3-2 exhibits strong phosphorus accumulation capacity, and the bacterial composition including the same is suitable for the treatment of phosphorus-rich domestic sewage in high-altitude mountainous regions, demonstrating the potential as a biological treatment agent.

The Acinetobacter pittii strain FL3-2 can achieve phosphorus accumulation under solely aerobic conditions without complex aerobic/anaerobic sequencing batch processes.

When the Acinetobacter pittii strain FL3-2 is cultured in a nutrient broth medium under aerobic conditions for 48 h and then transferred to simulated domestic sewage, a total phosphorus concentration in the supernatant of the simulated domestic sewage decreases from 5 mg/L to 1.4 mg/L, 2.1 mg/L and 2.4 mg/L after 96 h, yielding a mean phosphorus removal efficiency of 60.67%.

### BRIEF DESCRIPTION OF THE DRAWINGS

FIG. 1 shows a phylogenetic tree of an Acinetobacter pittii strain FL3-2 in accordance with an embodiment of the present disclosure;

FIGS. 2a-b show growth of the Acinetobacter pittii strain FL3-2 provided herein in a phosphorus-limited medium;

FIGS. 3a-b show growth of the Acinetobacter pittii strain FL3-2 provided herein in a phosphorus-excessive medium; and

FIG. 4 shows a standard curve of a phosphorus standard solution in accordance with an embodiment of the present disclosure.

### DETAILED DESCRIPTION OF EMBODIMENTS

Unless otherwise specified, materials and reagents used in the present disclosure are commercially available.

The Acinetobacter pittii strain FL3-2 provided by the disclosure has been deposited in the China Center for Type Culture Collection (Wuhan University, Wuhan City, Hubei Province, China (Postal Code: 430072)) on Dec. 21, 2023, with the deposit number of CCTCC NO: M20232618.

### EXAMPLE

### Step (1) Strain Isolation and Purification

During the research process, a bacterial strain was isolated from a soil sample obtained from Baima Snow Mountain (elevation: 4,000 m) in Deqin County, Diqing Prefecture, Yunnan Province, using a dilution spread plating technique and a streak plate method.

### Step (2) Strain Identification

The total deoxyribonucleic acid (DNA) of the bacterial strain was extracted as a template, and the 16S ribosomal ribonucleic acid (rRNA) was amplified with 27F (5′-AGAGTTTGATCCTGGCTCAG-3′ (SEQ ID NO:2)) as a forward primer and 1492R (5′-TACGGCTACCTTGTTACGACTT-3′ (SEQ ID NO:3)) as a reverse primer.

An amplified product was extracted and sequenced. The nucleotide sequence of the amplified product consists of SEQ ID NO: 1:

AAACTCCCATGGTGTGACGGGCGGTGTGTACAAGGCCCGGGAACGTATTC
ACCGCGGCATTCTGATCCGCGATTACTAGCGATTCCGACTTCATGGAGTC
GAGTTGCAGACTCCAATCCGGACTACGATCGGCTTTTTGAGATTAGCATC
CTATCGCTAGGTAGCAACCCTTTGTACCGACCATTGTAGCACGTGTGTAG
CCCTGGCCGTAAGGGCCATGATGACTTGACGTCGTCCCCGCCTTCCTCCA
GTTTGTCACTGGCAGTATCCTTAAAGTTCCCGACATTACTCGATGGCAAA
TAAGGAAAAGGGTTGCGCTCGTTGCGGGACTTAACCCAACATCTCACGAC
ACGAGCTGACGACAGCCATGCAGCACCTGTATGTAAGTTCCCGAAGGCAC
CAATCCATCTCTGGAAAGTTCTTACTATGTCAAGGCCAGGTAAGGTTCTT
CGCGTTGCATCGAATTAAACCACATGCTCCACCGCTTGTGCGGGCCCCCG
TCAATTCATTTGAGTTTTAGTCTTGCGACCGTACTCCCCAGGCGGTCTAC
TTATCGCGTTAGCTGCGCCACTAAAGCCTCAAAGGCCCCAACGGCTAGTA
GACATCGTTTACGGCATGGACTACCAGGGTATCTAATCCTGTTTGCTCCC
ATGCTTTCGCACCTCAGCGTCAGTGTTAGGCCAGATGGCTGCCTTCGCCA
TCGGTATTCCTCCAGATCTCTACGCATTTCACCGCTACACCTGGAATTCT
ACCATCCTCTCCCACACTCTAGCTAACCAGTATCGAATGCAATTCCCAAG
TTAAGCTCGGGGATTTCACATTTGACTTAATTAGCCGCCTACGCGCGCTT
TACGCCCAGTAAATCCGATTAACGCTTGCACCCTCTGTATTACCGCGGCT
GCTGGCACAGAGTTAGCCGGTGCTTATTCTGCGAGTAACGTCCACTATCT
CTAGGTATTAACTAAAGTAGCCTCCTCCTCGCTTAAAGTGCTTTACAACC
ATAAGGCCTTCTTCACACACGCGGCATGGCTGGATCAGGCTTGCGCCCAT
TGTCCAATATTCCCCACTGCTGCCTCCCGTAGGAGTCTGGGCCGTGTCTC
AGTCCCAGTGTGGCGGATCATCCTCTCAGACCCGCTACAGATCGTCGCCT
TGGTAGGCCTTTACCCCACCAACTAGCTAATCCGACTTAGGCTCATCTAT
TAGCGCAAGGTCCGAAGATCCCCTGCTTTCTCCCGTAGGACGTATGCGGT
ATTAGCATTCCTTTCGAAATGTTGTCCCCCACTAATAGGCAGATTCCTAA
GCATTACTCACCCGTCCGCCGCTAAGATCAGTAGCAAGCTACCTCTCTCC
GCTC.

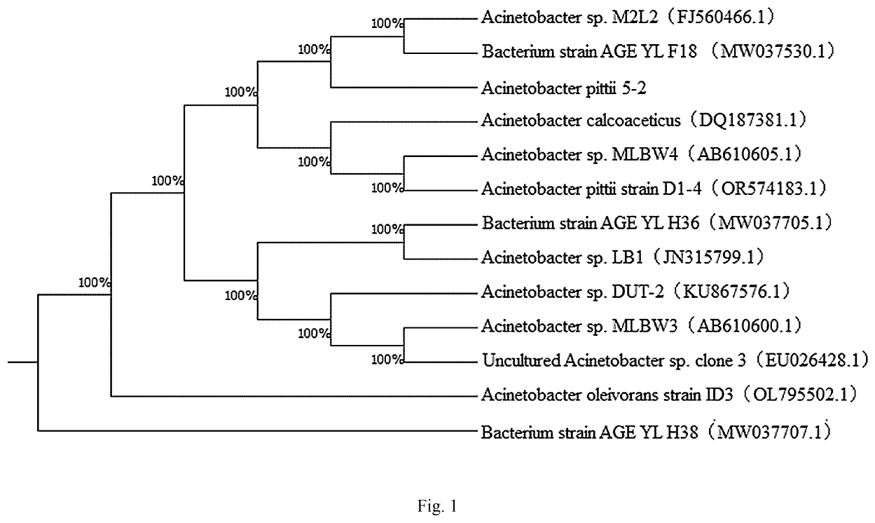

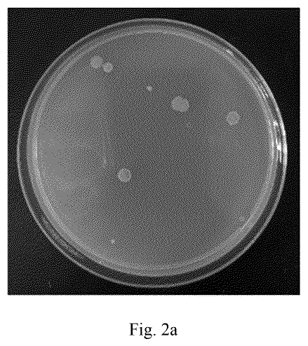

The obtained sequence was input into the National Center for Biotechnology Information (NCBI) website for comparison to identify related sequences, and similarity values were calculated. Finally, a phylogenetic tree was constructed using Molecular Evolutionary Genetics Analysis, version 11.0 (MEGA® 11.0), as shown in FIG. 1. The isolated strain was identified as the Acinetobacter pittii strain.

### Experimental Example Identification of Phosphorus Accumulation Capability

### 1. Blue-White Spot Screening

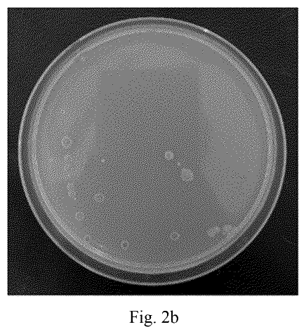

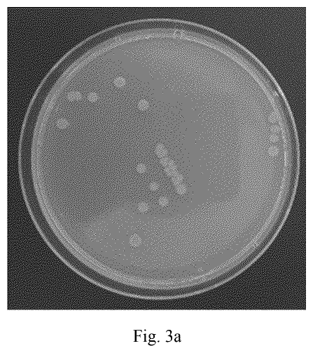

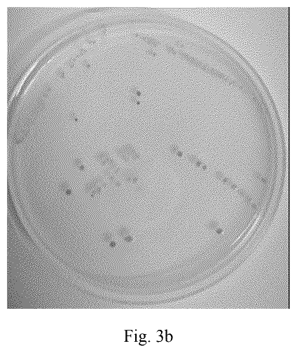

Under a sterile condition, the isolated strain was inoculated onto filter-sterilized 5-bromo-4-chloro-3-indolyl phosphate (BCIP) phosphorus-limited solid medium and a BCIP phosphorus-excessive solid medium using an inoculation loop, and corresponding codes and inoculation time were recorded. The cultures were incubated at 15° C. for 3 days, and changes of the inoculated strains were observed regularly. The strains displaying blue coloration on both the BCIP phosphorus-limited solid medium and the BCIP phosphorus-excessive solid medium were identified as polyphosphate-accumulating strains, that is, the primary screened strains of the present disclosure. It should be noted that the blue-white spot screening can only confirm that the screened strains have phosphorus accumulation capability, but cannot directly reflect the strength of phosphorus accumulation, which requires further quantification. Results of blue-white spot screening were shown in Table 1. The strains cultured for 48 h were shown in FIGS. 2a-b and 3a-b.

TABLE 1
Results of blue-white spot screening

| | Phosphorus- | Phosphorus- |
|---|---|---|
| Time to turn blue/h | limited medium | excessive medium |
| 24 | − | − |
| 48 | + | + |

Note:
“+” means turning blue;
“−” means not turning blue

### 2. Determination of Phosphorus Accumulation Capability

Ammonium molybdate spectrophotometry was employed to quantify phosphate removal efficiency, with the phosphate removal rate as an indicator to evaluate the phosphorus accumulation capability of the Acinetobacter pittii strain.

A simulated domestic sewage liquid culture medium was prepared as follows. 5 g/L peptone, 1.5 g/L beef extract powder, 2.5 g/L NaCl and 0.022 g/L potassium dihydrogen phosphate were mixed to obtain the simulated domestic sewage liquid culture medium with an initial total phosphorus concentration of 5 mg/L.

18 g of nutrient broth medium was mixed with 1 L of distilled water, and dispensed into 250-mL conical flasks with 200 mL per flask. The nutrient broth mediums were sterilized at 121° C. and cooled. The polyphosphate strains stored at −80° C. was inoculated into the conical flasks, and incubated with shaking at 15° C. and 180 rpm for 48 h, so as to obtain seed liquids. Then, each of liquid culture mediums were inoculated with 6 mL of the seed liquid (at a volume ratio of 3% (seed liquid/simulated domestic sewage liquid culture medium)) in triplicate. Incubation was carried out with shaking at 15° C. and 180 rpm for 96 h, and the absorbance of the culture solution was measured, and then the phosphorus concentration was calculated based on the absorbance and a standard curve.

The phosphorus accumulation effect was shown in Table 2.

TABLE 2
Effect of phosphorus accumulation

| | Initial phosphorus | Phosphorus concentration | Phosphorus |
|---|---|---|---|
| Number | concentration | after 48 h | removal rate |
| 1 | 5 mg/L | 1.4 mg/L | 72% |
| 2 | 5 mg/L | 2.1 mg/L | 58% |
| 3 | 5 mg/L | 2.4 mg/L | 52% |

### 3. Plotting of Standard Curve

10 g of ascorbic acid was diluted to 100 mL with water to obtain a 10% ascorbic acid solution.

13 g of ammonium molybdate ((NH₄)₆Mo₇·4H₂O) was dissolved in 100 mL of water to obtain an ammonium molybdate solution. 0.35 g of potassium antimonyl tartrate (K(SbO)C₄H₄O₆·1/2H₂O) was dissolved in 100 mL of water to obtain a potassium antimonyl tartrate solution. The ammonium molybdate solution was slowly added to 30 mL of a (1+1) sulfuric acid solution (a volume ratio of sulfuric acid to water of 1:1) under constant stirring, and then the potassium antimonyl tartrate solution was added to obtain a molybdate solution.

0.2197±0.001 g of potassium dihydrogen phosphate (dried at 110° C. for 2 h and cooled in a desiccator) was dissolved in water and transferred to a 1000-mL volumetric flask. About 800 mL of water and 5 mL of the (1+1) sulfuric acid solution was added into the 1000 mL volumetric flask followed by dilution to the mark with water and mixing, so as to obtain a phosphorus standard stock solution with 50 μg of phosphorus (in terms of P) per liter.

10 mL of the phosphorus standard stock solution was accurately pipetted into a 250-mL volumetric flask, diluted to the mark with water followed by mixing to obtain a phosphorus standard working solution with 2 μg of phosphorus per mL. The phosphorus standard working solution was prepared on the day of use.

0, 0.5, 1, 3, 5, 10 and 15 mL of the phosphate standard working solutions (2 μg/mL) were respectively pipetted into colorimetric tubes (50 mL) and diluted to 50 mL with distilled water, so as to obtain standard curve solutions with concentrations of 0, 0.02, 0.04, 0.12, 0.2, 0.4 and 0.6 μg/mL, respectively.

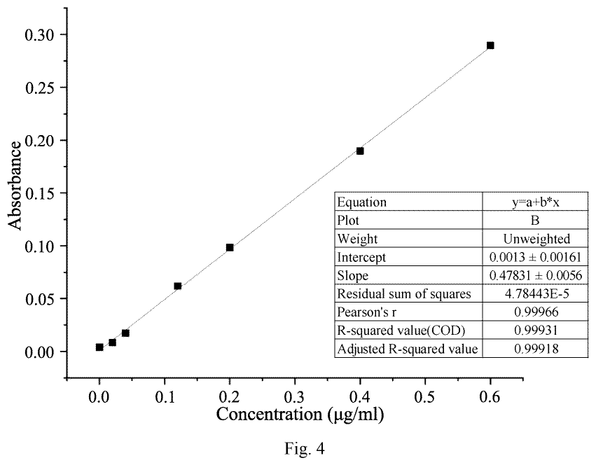

The standard curve was shown in FIG. 4.

Color development: 1 mL of the 10% ascorbic acid solution was added to a colorimetric tube, mixed with 2 mL of the molybdate solution after 30 s followed by standing for 15 min.

Measurement: The absorbance at a wavelength of 700 nm was measured using a micro-cuvette, with a zero-concentration solution as the reference.

Sample determination: The simulated domestic sewage culture solution containing the strain liquid was centrifuged at 12,000 rpm for 5 min to obtain a supernatant. 5 mL of the supernatant was transferred into a 50-mL volumetric flask, diluted to the fixed volume with water, and mixed with 1 mL of the 10% ascorbic acid solution. After 30 s, 2 mL of the molybdate solution was added followed by mixing. After the mixture was allowed to stand for 15 min, the absorbance was measured.

The phosphorus removal rate was calculated through the following equation: phosphorus removal rate=[(P₀−Pᵢ) Pₒ]×100%, where Pₒ represents a total phosphorus content after culturing without inoculation of the seed liquid of the Acinetobacter pittii strain, and Pᵢ represents a total phosphorus content after culturing with inoculation of the seed liquid of the Acinetobacter pittii strain.

It should be noted that the embodiments described above are merely illustrative of the present application, and are not intended to limit the scope of the present application. Although detailed descriptions have been made with reference to the above embodiments, various modifications and replacements made by those of ordinary skill in the art without departing from the spirit of the disclosure shall fall within the scope of the disclosure defined by the appended claims.

What is claimed is:

1. An Acinetobacter pittii strain, wherein the Acinetobacter pittii strain is Acinetobacter pittii FL3-2 with a deposit number of CCTCC NO: M20232618.

2. A bacterial composition, comprising:
the Acinetobacter pittii strain of claim 1 as an active ingredient.

3. An Acinetobacter pittii strain of claim 1, and/or
An application of the bacterial composition of claim 2 in the phosphorus removal.


SEQUENCE LISTING (3 sequences)

SEQ ID NO: 1 - DNA, 1354 bp, synthetic construct
  source 1..1354: mol_type = other DNA
aaactcccatggtgtgacgggcggtgtgtacaaggcccgggaacgtattcaccgcggcattctgatccgcgattactagcgattccgacttcatggagtcgagttgcagactccaatccggactacgatcggctttttgagattagcatcctatcgctaggtagcaaccctttgtaccgaccattgtagcacgtgtgtagccctggccgtaagggccatgatgacttgacgtcgtccccgccttcctccagtttgtcactggcagtatccttaaagttcccgacattactcgatggcaaataaggaaaagggttgcgctcgttgcgggacttaacccaacatctcacgacacgagctgacgacagccatgcagcacctgtatgtaagttcccgaaggcaccaatccatctctggaaagttcttactatgtcaaggccaggtaaggttcttcgcgttgcatcgaattaaaccacatgctccaccgcttgtgcgggcccccgtcaattcatttgagttttagtcttgcgaccgtactccccaggcggtctacttatcgcgttagctgcgccactaaagcctcaaaggccccaacggctagtagacatcgtttacggcatggactaccagggtatctaatcctgtttgctcccatgctttcgcacctcagcgtcagtgttaggccagatggctgccttcgccatcggtattcctccagatctctacgcatttcaccgctacacctggaattctaccatcctctcccacactctagctaaccagtatcgaatgcaattcccaagttaagctcggggatttcacatttgacttaattagccgcctacgcgcgctttacgcccagtaaatccgattaacgcttgcaccctctgtattaccgcggctgctggcacagagttagccggtgcttattctgcgagtaacgtccactatctctaggtattaactaaagtagcctcctcctcgcttaaagtgctttacaaccataaggccttcttcacacacgcggcatggctggatcaggcttgcgcccattgtccaatattccccactgctgcctcccgtaggagtctgggccgtgtctcagtcccagtgtggcggatcatcctctcagacccgctacagatcgtcgccttggtaggcctttaccccaccaactagctaatccgacttaggctcatctattagcgcaaggtccgaagatcccctgctttctcccgtaggacgtatgcggtattagcattcctttcgaaatgttgtcccccactaataggcagattcctaagcattactcacccgtccgccgctaagatcagtagcaagctacctctctccgctc

SEQ ID NO: 2 - DNA, 20 bp, synthetic construct
  source 1..20: mol_type = other DNA
agagtttgatcctggctcag

SEQ ID NO: 3 - DNA, 22 bp, synthetic construct
  source 1..22: mol_type = other DNA
tacggctaccttgttacgactt